(19) United States
(12) Patent Application Publication
(10) Pub. No.: US 2022/0296493 A1
(43) Pub. Date: Sep. 22, 2022

(54) PARTICULARLY NATURAL OIL SUBSTITUTE FOR COSMETIC PREPARATIONS

(71) Applicant: BEIERSDORF AG, Hamburg (DE)

(72) Inventors: Kerstin SKUBSCH, Prisdorf (DE); Kerstin FRANCK, Quickborn (DE); Nadine VOIGT, Hamburg (DE); Jette Mareike NEBEN, Hamburg (DE); Svea WISCHHOEFER, Hamburg (DE); Katharina HERWIG, Hamburg (DE); Katrin RUPP, Hamburg (DE)

(21) Appl. No.: 17/753,224

(22) PCT Filed: Aug. 21, 2020
(86) PCT No.: PCT/EP2020/073489

§ 371 Date: Feb. 24, 2022

(30) Foreign Application Priority Data
Aug. 27, 2019 (DE) 10 2019 212 793.2

(51) Int. Cl. A61K 8/73; A61K 8/37; A61K 8/06; A61Q 19/00

(52) U.S. Cl. CPC A61K 8/732; A61K 8/37; A61K 8/062; A61Q 19/007; A61K 2800/34

(57) ABSTRACT

Use of a mixture of a) hydroxypropyl starch phosphate (INCI: Hydroxypropyl Starch Phosphate) and b) esters of caprylic acid and/or capric acid as a substitute for mineral oils and silicone oils in cosmetic preparations and to their use in cosmetic preparations.

The present invention relates to a mixture of
a) hydroxypropyl starch phosphate (INCI: Hydroxypropyl Starch Phosphate) and
b) esters of caprylic acid and/or capric acid
   as a substitute for mineral and silicone oils in cosmetic preparations and to the use thereof in cosmetic preparations.

The desire to look beautiful and attractive is by nature ingrained in humans. Even though the ideal of beauty has undergone changes over time, people have always strived to have an immaculate appearance. An essential element of a beautiful and attractive appearance is the condition and appearance of the skin.

In order for the skin to perform its biological functions in full, it needs regular cleansing and care. Skin-care products, usually creams, ointments or lotions, are mostly used to moisturize the skin and replenish its natural oils. Often added to them are active substances that regenerate the skin and are intended, for example, to prevent and reduce its premature aging (e.g. formation of lines and wrinkles).

In addition to the cleansing and care of the skin, cosmetics have an aesthetic role too. They are intended to “improve” the external appearance of the user in accordance with the prevailing cultural perceptions. Cosmetics thus perform a psychological-social function, since they increase the (visual) attractiveness of users. First and foremost among them are “decorative” cosmetics that change the appearance of the user with the aid of coloring agents applied to the skin. However, cleansing and care products have an indirectly beneficial effect too, since a clean, healthy skin is the beauty ideal in humans.

Recent times have seen an increasing trend towards “natural” cosmetics, whose ingredients should ideally no longer derive from crude oil products or be chemically synthesized. This trend nowadays overlaps with the trend towards “vegan” products. The search for alternative ingredients meeting these criteria poses particular challenges to product developers. This is because substitution of known ingredients such as mineral oils, silicone oils and polyacrylates is practically always associated with disadvantages in terms of the use of the products. The products become unattractive from a sensory point of view which becomes unpleasantly apparent for example during spreading of the preparation on the skin and through inadequate absorbability. For instance the substitution of mineral oils, silicone oils and polyacrylates such as Carbomers, Acrylates/C10-30 Alkyl Acrylate Crosspolymer results in increased “whitening” when spreading the preparation onto the skin. This “whitening” occurs especially in preparations where the often employed xanthan gum (INCI: Xanthan Gum) is used instead of polyacrylates. Furthermore, the use of xanthan gum causes a stringy texture to occur, i.e. the formula forms strings when pulled apart between the fingers.

It is accordingly an object of the present invention to find a substitute for mineral oils and silicone oils in cosmetic preparations which has the same sensory properties (especially in terms of whitening, spreadability on the skin and absorbability) as the mineral and silicone oils.

It is a further object of the present invention to develop a cosmetic preparation (in particular an emulsion, here an O/W emulsion) which is free from mineral oils, silicone oils and polyacrylates which has the same sensory properties as conventional cosmetics containing these ingredients. These preparations should ideally be “vegan”, i.e. do without ingredients of animal origin. It was further sought to develop a preparation whose ingredients are of a vegetable origin, wherein vegetable origin is to be understood as also including substances whose starting material is of vegetable origin and which are subsequently subjected to one or more derivatization steps (for example hydrolysis, esterification).

The objects were surprisingly achieved through the use of a mixture of
a) hydroxypropyl starch phosphate (INCI: Hydroxypropyl Starch Phosphate) and
b) esters of caprylic acid and/or capric acid
   as a substitute for mineral and silicone oils in cosmetic preparations.

The objects were further achieved by a cosmetic preparation containing a mixture of
a) hydroxypropyl starch phosphate (INCI: Hydroxypropyl Starch Phosphate) and
b) esters of caprylic acid and/or capric acid, wherein the preparation is free from mineral oils, silicone oils, polyacrylates and xanthan gum.

It is advantageous according to the invention when the weight ratio of
a) hydroxypropyl starch phosphate to
b) the total amount of esters of caprylic acid/capric acid is from 1:1 to 1:20 and especially preferably from 1:2 to 1:15.

In the context of the present disclosure, the term “according to the invention” always relates to preparations and uses according to the invention unless otherwise stated.

Hydroxypropyl starch phosphates as described in U.S. Pat. No. 6,248,338 are especially advantageous. The use of a hydroxypropyl di-starch phosphate, such as is commercially available from Nouryon as the product Structure® XL, is very particularly preferred.

The embodiments of the present invention that are advantageous according to the invention are characterized in that the esters of caprylic acid and/or capric acid are selected from the group of the compounds caprylic acid/capric acid triglycerides (INCI: Caprylic/Capric Triglyceride), caprylyl caprylate/caprate (INCI: Caprylyl Caprylate/Caprate), coco caprylate/caprate (INCI: Coco Caprylate/Caprate), coco glycerides (INCI: Coco Glycerides), ethylhexyl cocoate (INCI; Ethyl Hexyl Cocoate), decyl cocoate (INCI: Decyl Cocoate), isoamyl cocoate (INCI: Isoamyl Cocoate), dicaprylyl carbonate (INCI: Dicaprylyl Carbonate), coco caprylate (INCI: Coco-Caprylate).

It is advantageous according to the invention for the preparation according to the invention when the preparation contains the hydroxypropyl starch phosphate (INCI: Hydroxypropyl Starch Phosphate) in a concentration of 0.2% to 5% by weight based on the total weight of the preparation.

The preparations according to the invention are moreover advantageously characterized in that the preparation is in the form of an emulsion.

In such a case it is preferable according to the invention when the preparation is in the form of an oil-in-water emulsion (O/W emulsion).

In such a case the embodiments particularly preferred according to the invention are characterized in that the preparation contains one or more emulsifiers selected from the group of the compounds glyceryl stearate citrate (INCI: Glyceryl Stearate Citrate), sodium stearoyl glutamate (INCI: Sodium Stearoyl Glutamate), glyceryl stearate SE (INCI: Glyceryl Stearate SE), sodium cetearyl sulfate (INCI: Sodium Cetearyl Sulfate), stearic acid (INCI: Palmitic Acid+Stearic Acid+Myristic Acid+Arachidic Acid+Oleic Acid), Glyceryl Stearate (INCI: Glyceryl Stearate), Polyglyceryl-3 diisostearate (INCI: Polyglyceryl-3 Diisostearate).

Here it is advantageous according to the invention when the emulsifiers are employed in a usage concentration of 0.1% to 5% by weight based on the total weight of the preparation.

The embodiments of the present invention that are advantageous according to the invention are further characterized in that the preparation is free from polyethylene glycol ethers and esters and free from parabens, methylisothiazolinone, chloromethylisothiazolinone and DMDM hydantoin.

The preparation according to the invention should moreover preferably be free from animal constituents.

It is advantageous in the context of the present invention when the preparation contains waxes and/or fatty alcohols. According to the invention it is preferable to employ waxes/fatty alcohols from the list of the following compounds: Cetearyl Alcohol, Cetyl Alcohol, Stearyl Alcohol, Butyrospermum Parkii Butter, Cetyl Palmitate, Methyl Palmitate, Myristyl Myristate, Hydrogenated Coco-Glycerides, Myristyl Alcohol, Behenyl Alcohol, Hydrogenated Castor Oil, Theobroma Cacao Seed Butter, Hydrogenated Rapeseed Oil, Caprylic/Capric/Myristic/Stearic Triglyceride, Hydrogenated Vegetable Oil, Carnauba Wax, Candelilla Wax, Sunflower Wax, Tristearin.

In addition the embodiments of the present invention that are advantageous according to the invention are characterized in that the preparation contains additional vegetable oils selected from the group of Persea Gratissima Oil, Orbignya Oleifera Seed Oil, Argania Spinosa Kernel Oil, Prunus Armeniaca Kernel Oil, Simmondsia Chinensis Seed Oil, Cocos Nucifera Oil, Silybum Marianum Seed Oil, Oenothera Biennis Oil, Olea Europaea Fruit Oil, Helianthus Annuus Seed Oil, Vitis Vinifera Seed Oil, Cannabis Sativa Seed Oil, Olus Oil, Vegetable Oil, Gossypium Herbaceum Seed Oil, Arctium Lappa Seed Oil, Macadamia Ternifolia Seed Oil, Macadamia Integrifolia Seed Oil, Zea Mays Germ Oil, Prunus Amygdalus Dulcis Oil, Ricinus Communis Seed Oil, Glycine Soja Oil, Helianthus Annuus Hybrid Oil, Sesamum Indicum Seed Oil, Brassica Campestris Seed Oil.

It was moreover found that it is of particular advantage according to the invention when the preparation contains one or more lipophilic components selected from the group of the compounds Dicaprylyl Ether, Decyl Oleate, Octyldodecanol, Squalane, Triisostearin, Shea Butter Ethyl Ester, Triheptanoin, Hexyldecyl Stearate, Isoamyl Laurate.

Advantageous embodiments of the present invention are also characterized in that the preparation contains one or more alkanediols from the group of the compounds 1,2-pentanediol, 1,2-hexanediol, 1,2-octanediol, 1,2-decanediol, 2-methyl-1,3-propanediol, ethylhexylglycerin.

Embodiments of the present invention that are advantageous according to the invention are characterized in that the preparation contains one or more compounds selected from the group of the compounds alpha-lipoic acid, folic acid, phytoene, D-biotin, coenzyme Q10, alpha-glucosylrutin, carnitine, carnosine, natural isoflavonoids, flavonoids, creatine, creatinine, taurine, β-alanine, tocopherol, panthenol, magnolol, honokiol, glycerylglycose, hyaluronic acid and/or salts thereof and/or licochalcone A.

The preparations according to the invention may moreover contain further cosmetic ingredients.

It is therefore advantageous when the preparation contains fillers, in particular tapioca starch. The usage concentration for tapioca starch advantageous according to the invention is 0.05% to 3.0% by weight based on the total weight of the preparation. Tapioca starch has the advantage over other fillers, such as for example zinc oxide, that the preparation does not become too hard.

The water phase of the preparations according to the invention may advantageously contain customary cosmetic auxiliaries, for example alcohols, in particular those having a low carbon number, preferably ethanol.

### Comparative Experiment

The following experiment demonstrated the effect according to the invention: The following preparations were produced and their whitening effect upon spreading onto the skin was determined.

| | A | B | C |
|---|---|---|---|
| INCI | m [%] | m [%] | m [%] |
| Sodium Cetearyl Sulfate | 0.15 | 0.15 | 0.15 |
| Glyceryl Stearate SE | 1.5 | 1.5 | 1.5 |
| Glyceryl Stearate | 1 | 1 | 1 |
| Cetearyl Alcohol | 4 | 4 | 4 |
| Butyrospermum Parkii Butter | 1 | 1 | |
| Cocoglyceride | 6 | 6 | 6 |
| Coco-Caprylate | 4 | 4 | 4 |
| Carbomer (=polyacrylat) | 0.5 | | |
| Hydroxypropyl Starch Phosphate | | 0.5 | |
| Xanthan Gum | | | 0.5 |
| Glycerin | 7 | 7 | 7 |
| Phenoxyethanol | 0.9 | 0.9 | 0.9 |
| Ethylhexylglycerin | 0.2 | 0.2 | 0.2 |
| 45% sodium hydroxide soln. | 0.06 | | |
| Aqua | ad 100 | ad 100 | ad 100 |
| Whitening effect upon | strong | faint | very |
| spreading onto the underarm | | | strong |
| area (75 μl in each case) | | | |

### EXAMPLES

The following examples are intended to elucidate the present invention without limitation thereof. All reported amounts, proportions and percentages are based on the weight and the total amount/on the total weight of the preparations unless otherwise stated.

| INCI | m [%] | m [%] | m [%] | m [%] |
|---|---|---|---|---|
| | D | E | F | G |
| Hydroxypropyl Starch Phosphate | 0.7 | 1 | 2.5 | 1.5 |
| Cocoglyceride | 3 | | | |
| Decyl Cocoate | | | 3 | 3 |
| Coco Caprylate/Caprate | 3 | | | |
| Dicaprylylether | 1.5 | | | |
| Caprylic/Capric Triglyceride | | 3 | | |
| Shea Butter Ethyl Ester | | | 3 | |
| Prunus Amygdalus Dulcis Oil | | 2 | | |
| Cocos Nucifera Oil | | | 0.5 | |
| Theobroma Cacao Seed Butter | | | 1 | |
| Sodium Cetearyl Sulfate | 0.15 | 0.15 | 0.2 | |
| Sodium Stearoyl Glutamate | | | | 0.2 |
| Glyceryl Stearate SE | 2 | 2 | 2.5 | |
| Glyceryl Stearate | 1 | | 0.5 | 2 |
| Cetearyl Alcohol | 4 | 2 | | |
| Butyrospermum Parkii Butter | 1 | | | |
| Cetyl Palmitate | | 1 | | |
| Glycerin | 7 | 7 | 5 | 8 |
| Phenoxyethanol | 0.5 | | | |
| Ethylhexylglycerin | 0.5 | 0.3 | | |
| Benzylalcohol | | 0.2 | | |
| Polyglyceryl-2 Caprate | | | 0.5 | |
| Alcohol | 3 | | 4 | |
| Perfume | 0.4 | | 0.3 | 0.2 |
| Aqua | balance | balance | balance | balance |
| | to 100 | to 100 | to 100 | to 100 |
| | H | I | K | L |
| Hydroxypropyl Starch Phosphate | 1 | 2 | 0.5 | 1.25 |
| Caprylic/Capric Triglyceride | 3 | 3 | 1 | |
| Octyldodecanol | 2 | | 0.2 | 0.5 |
| Ethylhexylcocoat | | 3 | | 1 |
| Decylcocoat | 2 | | | 1 |
| Helianthus Annuus Hybrid Oil | | 2 | | |
| Vegetable Oil | | | 0.3 | 0.9 |
| Triisostearin | | | 1.2 | 1.5 |
| Glyceryl Stearate Citrate | 1.5 | 2 | | |
| Palmitic Acid + Stearic Acid + | | | 0.8 | 2 |
| Myristic Add + Arachidic Acid + | | | | |
| Oleic Acid | | | | |
| Glyceryl Stearate | | 1 | 1 | 0.8 |
| Butyrospermum Parkii Butter | | | | |
| Cetearyl Alcohol | 4 | 3 | | |
| Myristyl Alcohol | | | 5 | |
| Cetylalkohol | | | | 3.5 |
| Glycerin | 7 | 5 | 4 | 5 |
| Sorbic Acid | 0.1 | 0.2 | | |
| Tocopherol | 0.2 | | | |
| Ethylhexylglycerin | | | 0.2 | 0.5 |
| Benzoic Acid | | 0.2 | | |
| Alcohol | | | 5 | 3 |
| Perfume | 0.3 | | 0.15 | 0.4 |
| Aqua | balance | balance | balance | balance |
| | to 100 | to 100 | to 100 | to 100 |

1.-15. (canceled)

16. A cosmetic preparation, wherein the preparation comprises a mixture of
(a) hydroxypropyl starch phosphate and
(b) esters of caprylic acid and/or capric acid,
the preparation being free from mineral oils, silicone oils, polyacrylates and xanthan gum.

17. The preparation of claim 16, wherein a weight ratio (a):(b) is from 1:1 to 1:20.

18. The preparation of claim 16, wherein a weight ratio (a):(b) is from 1:2 to 1:15.

19. The preparation of claim 16, wherein (b) is selected from one or more of caprylic acid/capric acid triglycerides (INCI: Caprylic/Capric Triglyceride), caprylyl caprylate/caprate (INCI: Caprylyl Caprylate/Caprate), coco caprylate/caprate (INCI: Coco Caprylate/Caprate), coco glycerides (INCI: Coco Glycerides), ethylhexyl cocoate (INCI: Ethyl Hexyl Cocoate), decyl cocoate (INCI: Decyl Cocoate), isoamyl cocoate (INCI: Isoamyl Cocoate), dicaprylyl carbonate (INCI: Dicaprylyl Carbonate), coco caprylate (INCI: Coco-Caprylate).

20. The preparation of claim 16, wherein the preparation contains (a) in a concentration of from 0.2% to 5% by weight based on a total weight of the preparation.

21. The preparation of claim 16, wherein the preparation is in the form of an emulsion.

22. The preparation of claim 16, wherein the preparation is in the form of an oil-in-water (O/W) emulsion.

23. The preparation of claim 16, wherein the preparation is free from polyethylene glycol ethers and esters and free from parabens, methylisothiazolinone, chloromethylisothiazolinone and DMDM hydantoin.

24. The preparation of claim 16, wherein the preparation further comprises waxes and/or fatty alcohols.

25. The preparation of claim 16, wherein the preparation further comprises one or more vegetable oils selected from Persea Gratissima Oil, Orbignya Oleifera Seed Oil, Argania Spinosa Kernel Oil, Prunus Armeniaca Kernel Oil, Simmondsia Chinensis Seed Oil, Cocos Nucifera Oil, Silybum Marianum Seed Oil, Oenothera Biennis Oil, Olea Europaea Fruit Oil, Helianthus Annuus Seed Oil, Vitis Vinifera Seed Oil, Cannabis Sativa Seed Oil, Olus Oil, Vegetable Oil, Gossypium Herbaceum Seed Oil, Arctium Lappa Seed Oil, Macadamia Ternifolia Seed Oil, Macadamia Integrifolia Seed Oil, Zea Mays Germ Oil, Prunus Amygdalus Dulcis Oil, Ricinus Communis Seed Oil, Glycine Soja Oil, Helianthus Annuus Hybrid Oil, Sesamum Indicum Seed Oil, Brassica Campestris Seed Oil.

26. The preparation of claim 16, wherein the preparation further comprises one or more emulsifiers selected from glyceryl stearate citrate (INCI: Glyceryl Stearate Citrate), sodium stearoyl glutamate (INCI: Sodium Stearoyl Glutamate), glyceryl stearate SE (INCI: Glyceryl Stearate SE), sodium cetearyl sulfate (INCI: Sodium Cetearyl Sulfate), stearic acid (INCI: Palmitic Acid+Stearic Acid+Myristic Acid+Arachidic Acid+Oleic Acid), Glyceryl Stearate (INCI: Glyceryl Stearate), Polyglyceryl-3 diisostearate (INCI: Polyglyceryl-3 Diisostearate).

27. The preparation of claim 26, wherein the one or more emulsifiers are present in a concentration of from 0.1% to 5% by weight based on a total weight of the preparation.

28. The preparation of claim 16, wherein the preparation is free from animal derived constituents.

29. The preparation of claim 16, wherein the preparation further comprises one or more lipophilic components selected from dicaprylyl ether, decyl oleate, octyldodecanol, squalane, triisostearin, shea butter ethyl ester, triheptanoin, hexyldecyl stearate, isoamyl laurate.

30. The preparation of claim 16, wherein the preparation further comprises one or more alkanediols selected from 1,2-pentanediol, 1,2-hexanediol, 1,2-octanediol, 1,2-decanediol, 2-methyl-1,3-propanediol, ethylhexylglycerin.

31. The preparation of claim 16, wherein the preparation further comprises one or more fillers.

32. The preparation of claim 31, wherein the one or more fillers comprise tapioca starch.

33. The preparation of claim 32, wherein the tapioca starch is present in a concentration of from 0.05% to 3.0% by weight based on a total weight of the preparation.

34. The preparation of claim 16, wherein the preparation further comprises ethanol.

35. A method of substituting mineral and silicone oils in a cosmetic preparation, wherein the method comprises using a mixture of hydroxypropyl starch phosphate and esters of caprylic acid and/or capric acid instead of the mineral and silicone oils.
